(19) United States
(12) Patent Application Publication
(10) Pub. No.: US 2022/0396764 A1
(43) Pub. Date: Dec. 15, 2022

(54) MATRICES FOR CELL CULTURE

(71) Applicant: AGENCY FOR SCIENCE, TECHNOLOGY AND RESEARCH, Singapore (SG)

(72) Inventors: Andrew Chwee Aun WAN, Singapore (SG); Du CHAN, Singapore (SG)

(21) Appl. No.: 17/776,164

(22) PCT Filed: Nov. 11, 2020
(86) PCT No.: PCT/SG2020/050649

§ 371 Date: May 11, 2022

(30) Foreign Application Priority Data
Nov. 12, 2019 (SG) 10201910571V

(51) Int. Cl. C12N 5/00; C07K 14/37

(52) U.S. Cl. CPC C12N 5/0068; C12N 5/0075; C07K 14/37; C12N 2533/72; C12N 2533/74; C12N 2533/78; C12N 2500/92

(57) ABSTRACT

There is provided a cell culture matrix comprising a fungal derived protein. Also provided is a composition comprising the cell culture matrix as described herein, a cell culture system comprising the cell culture matrix as described herein, and a method of forming a cell culture matrix thereof.

### TECHNICAL FIELD

The present disclosure relates broadly to matrices for cell cultures. The present disclosure relates to hydrogel or extracellular matrix gel for cell culture.

### BACKGROUND

The most widely used extracellular matrix (ECM) gels for 3D culture of mammalian cells are animal-derived ECM components such as collagen, laminin and fibronectin. A mouse osteosarcoma matrix, commercialized as Matrigel or Geltrex, is another popular material in use for in vitro cell culture studies. These matrices possess cell-adhesive motifs which mediate cell attachment. Synthetic hydrogels such as PEG diacrylate and hydrogels based on natural polymers such as sodium alginate are also commonly used, however, they have to be imbued with cell-adhesive moieties, which increases the cost and widespread applicability of these materials. One particular area of application where non animal-derived ECM alternatives are required is in the burgeoning Clean Meat industry, where a vegetal-derived gel would be desirable.

Thus, there is a need to provide extracellular matrix gels that are free from animal-derived components. There is a need to provide an alternative extracellular matrix gel.

### SUMMARY

In one aspect, there is provided a cell culture matrix comprising a fungal derived protein.

In some examples, the fungal derived protein comprises a cell-adhesive sequence, optionally, the fungal derived protein retains its primary sequence.

In some examples, the fungal derived protein is free of native functions. In some examples, the fungal derived protein forms particles having an average diameter of about 500 nm to about 1600 nm.

In some examples, the fungal derived protein forms particles having a zeta potential of about −10 mV to about −50 mV.

In some examples, the fungal derived protein extract forms a gel, optionally a hydrogel.

In some examples, the fungi are non-pathogenic fungi.

In some examples, the fungi are from the order of Agaricales, optionally from the division Basidiomycota.

In some examples, the fungi are species selected from the group consisting of Flammulina velutipes, Lentinus edodes, and Hypsizigus marmoreus.

In some examples, the cell culture matrix further comprises a biomaterial component, optionally the cell culture matrix further comprises one or more selected from the group consisting of alginate, chitosan, chitin, and cellulose fibres.

In some examples, the cell culture matrix further comprises crosslinkers, optionally the crosslinker is calcium.

In some examples, the cell culture matrix forms a coating comprising alginate that is calcium crosslinked.

In another aspect, there is provided a composition comprising the cell culture matrix as described herein.

In yet another aspect, there is provided a cell culture system comprising a cell culture matrix comprising a fungal derived protein.

In some examples, the cell culture system may comprise the cell culture matrix as described herein and/or the composition as described herein.

In yet another aspect, there is provided a method of forming a cell culture matrix from a fungus, comprising extracting a fungal derived protein from the fungi.

In some examples, the extraction comprises aggregation of the fungal derived protein to form self-aggregating precipitate.

In some examples, the extraction step comprises aggregation by boiling the fungal derived protein and/or incubating the fungal derived protein in a condition that allows self-aggregating precipitate.

In some examples, the extraction step comprises a fungal derived protein denaturation step that causes aggregation to occur, optionally wherein the denaturation step retains the primary sequence structure of the fungal derived protein, optionally the denaturation step retains the cell-adhesive sequence of the fungal derived protein.

In some examples, the extraction step comprises aggregation step comprising heat treating the fungal derived protein for about 1 min to about 30 mins, optionally the aggregation step comprises heating the fungal derived protein from about 50° C. to about 350° C.

### Definitions

As used herein, the term “aqueous” or “aqueous medium” refers to include water as a constituent.

As used herein, the term “matrix” or “matrices” refers to substance or natural material in which something (such as a cell) is embedded. Thus, in the present disclosure, “matrix” or “matrices” refers to a substance (typically extracellular substance) in which cells may be embedded and/or adhered to. Such “matrix” or “matrices” may support the survival and/or proliferation and/or differentiation and/or migration of a cell in vivo or in vitro. In other words, the “matrix” or “matrices” as described herein may support complex cellular behaviour that may not otherwise occur in a cell culture without its support. In some examples, the “matrix” or “matrices” as described herein may be useful as an attachment substrate.

As used herein, the term “hydrogel” refers to a three-dimensional, hydrophilic or amphiphilic polymeric network that holds together and comprises water as its major component (and/or is capable of taking up large quantities of water). As used herein, the networks are composed of polymers (such as polypeptides) and are insoluble due to the presence of covalent chemical or physical (ionic, hydrophobic interactions, entanglements) crosslinks. The crosslinks provide the network structure and physical integrity that is useful when used as a scaffold (such as bio-scaffold). Hydrogels exhibit a thermodynamic compatibility with water that allows them to swell in aqueous media. Thus, in some examples, the hydrogel as described herein may be used as extracellular matrix (ECM) gels for culture of cell lines in vitro. In some examples, the hydrogel as describe herein may be used as extracellular matrix to support 3-dimensional (3D) culture of cell lines (such as mammalian cells) in vitro.

As used herein, the term “gel” may also refer to “hydrogel”. In some examples, the term “gel” also refers to a dispersion where the dispersed phase has incorporated at least a portion of the dispersion medium to produce a solid or semi-solid, elastically deformable material.

As used herein, “RGD” motif relates to an example of a cell-adhesive sequence and/or motif that comprises the tripeptide amino acid sequence comprising Arg-Gly-Asp (RGD) or Arginine, Glycine, and Aspartate.

As used herein, “YF” motif relates to an example of a cell-adhesive sequence and/or motif that comprises the peptide amino acid sequence comprising Tyr-Phe (YF) or Tyrosine and Phenylalanine.

The term “micro” as used herein is to be interpreted broadly to include dimensions from about 1 micron to about 1000 microns.

The term “nano” as used herein is to be interpreted broadly to include dimensions less than about 1000 nm.

The term “particle” as used herein broadly refers to a discrete entity or a discrete body. The particle described herein can include an organic, an inorganic or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidal shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

The terms “coupled” or “connected” as used in this description are intended to cover both directly connected or connected through one or more intermediate means, unless otherwise stated.

As used herein the term “coat” describe the application of a layer of a substrate or other element of an article (such as hydrogels as described herein, hydrogels in the art, (additional/other) extracellular matrix (other than the fungal-derived hydrogel as described herein), and the like) as described herein. The layering/coating process as described herein may layer the hydrogel as described herein atop a substrate (such as other extracellular matrix) or other element, but not necessary contiguous to either the substrate or the other element. In some example, the hydrogel as described herein coats a chitosan microsphere. The term “atop”, “coating”, “on”, “over”, “uppermost”, “underlying” and the like for the location of various elements disclosed in the present disclosure refers to the relative position of an element with respect to a horizontally-disposed, upwardly facing substrate. However, unless otherwise indicated, it is not intended that the substrate or article should have any particular orientation in space during or after manufacture.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

The inventors of the present disclosure postulated that an inexpensive alternative for cell-adhesion matrices would be fungal-derived proteins. An RGD-containing adhesive protein, MFBA has been reported in the basidiomycete fungi, Lentinus Edodes, where it plays the postulated role of inducing hyphal cell aggregation. The recombinant form of MFBA, has been shown to support melanoma cell adhesion in culture. Similar proteins have also been found in Flammulina velutipes and postulated with similar functions. However, there have been no reports of isolation of fungal extracts for use in mammalian cell culture, or formulation of the cell-adhesive component of the extract into a material that could be used for the purpose.

Here, the present disclosure demonstrates the discovery and isolation of uniform protein particles from two species of fungi, Flammulina velutipes and Lentinus edodes, which belong to the order, Agaricales and division, Basidiomycota. Under suitable conditions, the particles can be induced to aggregate and form hydrogels, which the present disclosure shows to be useful for 3D mammalian cell culture. The extract of a third species belonging to the same order and family, Hypsizigus marmoreus, supported cell adhesion, but did not yield the same gel-forming protein particles.

As such, exemplary, non-limiting embodiments of fungal-derived cell-adhesive matrices are disclosed hereinafter.

In one aspect, there is provided a cell culture matrix comprising fungal derived protein. In some examples, the fungal derived protein may include, but is not limited to adhesin, collagen-binding protein and fasciclin (FAS1 domain). In some examples, the fungal derived protein may be collagen-binding protein and/or fasciclin (FAS1 domain). In some examples, the fungal derived protein comprises a cell-adhesive sequence and/or motif (such as an RGD motif and/or an YH motif), optionally, the fungal derived protein retains its primary sequence. The inventors of the present disclosure found that heating the extract, which is believed to cause protein denaturation and loss of higher level structures, advantageously retains the primary sequence that is required for cell-adhesion. Whilst not wishing to be bound by theory, it is believed that the loss of higher level structures is desirable because the protein would lose its native functions but not its primary sequence, which contains the cell-adhesive function of the RGD motif. Therefore, in some examples, the fungal derived protein is free of its native (non cell-adhesive) functions. In some examples the protein is free of (or has lost its) secondary and/or tertiary structure that confer the native function(s) of the protein.

In some examples, fungal derived protein forms particles having an average diameter of about 500 nm to about 1600 nm. In some examples, the fungal derived protein particles may have an average diameter of about 500 nm to about 1600 nm, or about 600 nm to about 1500 nm, or about 700 nm to about 1400 nm, or about 700 nm to about 1300 nm, or about 800 nm to about 1200 nm, or about 900 nm to about 1100 nm. In some examples, the fungal derived protein particles may have an average diameter of about 900 nm, or about 910 nm, or about 920 nm, or about 930 nm, or about 940 nm, or about 950 nm, or about 960 nm, or about 970 nm, or about 980 nm, or about 990 nm, or about 1000 nm, or about 1010 nm, or about 1020 nm, or about 1030 nm, or about 1040 nm, or about 1050 nm, or about 1060 nm, or about 1070 nm, or about 1080 nm, or about 1090 nm, or about 1100 nm, or about 1200 nm, or about 1300 nm, or about 1400 nm, or about 1500 nm. In some examples, the fungal derived protein particles may have an average diameter of about 960 nm or 1050 nm.

In some examples, the fungal derived protein forms particles having a zeta potential of about −10 mV to about −50 mV. In some examples, the fungal derived protein particles may have a zeta potential of about −10 mV, or about −11 mV, or about −12 mV, or about −13 mV, or about −14 mV, or about −15 mV, or about −16 mV, or about −17 mV, or about −18 mV, or about −19 mV, or about −20 mV, or about −21 mV, or about −22 mV, or about −23 mV, or about −24 mV, or about −25 mV, or about −26 mV, or about −27 mV, or about −28 mV, or about −29 mV, or about −30 mV, or about −31 mV, or about −32 mV, or about −33 mV, or about −34 mV, or about −35 mV, or about −36 mV, or about −37 mV, or about −38 mV, or about −39 mV, or about −40 mV, or about −41 mV, or about −42 mV, or about −43 mV, or about −44 mV, or about −45 mV, or about −46 mV, or about −47 mV, or about −48 mV, or about −49 mV, or about −50 mV. In some examples, the fungal derived protein particles may have a zeta potential of about −20 mV or about −28 mV.

In some examples, a suitable fungal species may be a fungus that yields protein particles that are sufficiently uniform and/or negatively charged upon denaturation (by boiling). Without wishing to be bound by theory, it is believed that the overall negative charge of the protein particles upon denaturation assist the particles in repelling each other and are thus easily dispersed in water. When the ionic strength is increased by adding a buffered saline (such as PBS), counter-ion screening of the particle surface charges occurs, thus leading to particle aggregation and network crosslinking formation (such as gel/hydrogel formation). Therefore, in some examples, a suitable fungal species may comprise protein particles being negatively charged when denatured.

In some examples, the fungi are non-pathogenic fungi. In some examples, the fungi are edible mushrooms, optionally edible gilled mushrooms. In some examples, a suitable fungus would be a fungus that comprises protein(s) that has a cell-adhesive sequence (such as RGD motif, and the like). In some examples, a suitable fungus would not include fungus that is known to be pathogenic (such as Candida albicans) to humans, animals, and/or plants. In some examples, the fungi are from the order of Agaricales, optionally from the division Basidiomycota. As exemplified in the present disclosure, fungi such as Flammulina velutipes, Lentinus edodes, and Hypsizigus marmoreus are suitable candidates for the cell culture matrix as described herein. Thus, in some examples, the fungi are species including, but not limited to, Flammulina velutipes, Lentinus edodes, Hypsizigus marmoreus, and the like. In some examples, the fungi may be Flammulina velutipes and Lentinus edodes.

As described in the Experimental Section, the cell culture matrix as described herein can be used in conjunction with other extracellular matrix as an overcoat or in bulk-incorporation. Thus, in some examples, the cell culture matrix may further comprise a biomaterial component, optionally the cell culture matrix may further comprise one or more biomaterial selected from the group consisting of alginate, chitosan, chitin, and cellulose fibres. In some examples, the cell culture matrix may further comprise an additional biomaterial component that, depending on the need, may have the same, substantially similar, and/or different property as the fungal-derived protein extract. For example, the cell culture matrix may further comprise alginate, chitosan, chitin, cellulose fibres, and the like. That is, in some examples, the cell culture matrix as described herein may undergo bulk-incorporation and/or coating of fungal derived protein particles (i.e. fungal-derived protein extract) in one or more biomaterial components, such as, but not limited to, alginate solution, chitosan spheres (or microspheres), (water soluble) chitin-alginate fibres, cellulose fibres, alginate spheres and alginate films.

As the fungal derived protein and/or cell culture matrix as described herein comprises high negative charge particles that aggregate upon increase in ionic strength, the fungal derived protein and/or cell culture matrix as described herein advantageously facilitates the formation of hydrogel network that is capable of immobilising and/or capturing and/or retaining and/or holding one or more binding molecules. Therefore, in some examples, the fungal derived protein and/or cell culture matrix may be used to form gels (such as particulate gels) and/or coating. For example, the cell culture matrix may be used to form gel/hydrogel. In some examples, the hydrogel may comprise porous stably crosslinked network that immobilises one or more binding molecules, which in turn can bind and/or crosslink and/or couple to one or more biological molecules.

In some example, the fungal derived protein and/or cell culture matrix as described herein may be used to coat other biomaterials. For example, the hydrogel may be used to coat microspheres, such as chitosan microspheres. In some examples, the cell culture matrix may further comprise a crosslinker. In some examples, the cell culture matrix may further comprise a crosslinker, such as divalent cations and/or organic crosslinkers. In some examples, the cell culture matrix may further comprise mineral crosslinking such as but is not limited to divalent cations such as calcium, barium and the like. In some examples, the crosslinker is calcium. In some examples, the cell culture matrix may further comprise an organic crosslinker such as genipin. In some examples, the cell culture matrix may further comprise minor crosslinking, optionally calcium crosslinking. In some examples, the cell culture matrix forms a coating comprising alginate (such as FV-alginate as described herein) that is calcium crosslinked (i.e. a calcium crosslinked alginate).

Binding molecules and/or biomaterials can bind and/or crosslink and/or be coupled to the hydrogel network via covalent and/or non-covalent binding. For example, covalent bonding may comprise chemically or covalently binding one or more binding molecule and/or biomaterial to the network. In some examples, non-covalent bonding may include, but is not limited to, ionic bonds, metal coordination bonds, hydrogen bonds, van der Waals forces, ion-dipole, dipole dipole, p-p stacking, hydrophobic interactions and the like.

Due to the versatile nature of the fungal derived protein as described herein, the cell culture matrix may be moulded into any form that would be useful for a person skilled in the art to culture cells. Therefore, the cell culture matrix as described herein may be provided in any form that is suitable for cell culture. Thus, the cell culture matrix as disclosed herein may be provided in a form such as, but is not limited to, substantially cylindrical or tubular (such as a fibre), substantially spherical (such as sphere/microspheres), film, non-regular shapes (irregular shaped or ellipsoid shaped particles) and the like. In some examples, the hydrogel as described herein may be the building block of various biomaterials. For example, the cell culture matrix as described herein may be used in myotube formation as it can advantageously promote muscle cell alignment. For example, the cell culture matrix as described herein may be provided as a hydrogel that is made in to fibers or other suitable forms with high aspect ratio that can align muscle cells. In some examples, the alignment of muscle cells as assisted by the cell culture matrix as described herein allows the differentiation of muscle cells into myotubes and/or myofibers.

In another aspect, there is provided a composition comprising the cell culture matrix as described herein.

In yet another aspect, there is provided a composition comprising the cell culture system having fungal derived protein as described herein (such as fungal derived protein particles or extract). In some examples, the cell culture system as described herein comprising the cell culture matrix as described herein and/or the composition as described herein.

In some examples, the cell cultures system comprises the fungal derived protein as described herein (such as fungal derived protein particles or extract) in a concentration of about 1 mg/ml, or about 1.1 mg/ml, or about 1.2 mg/ml, or about 1.3 mg/ml, or about 1.4 mg/ml, or about 1.5 mg/ml, or about 1.6 mg/ml, or about 1.7 mg/ml, or about 1.8 mg/ml, or about 1.9 mg/ml, or about 2 mg/ml, or about 3 mg/ml, or about 4 mg/ml, or about 5 mg/ml, or about 6 mg/ml, or about 7 mg/ml, or about 8 mg/ml, or about 9 mg/ml, or about 10 mg/ml, or about 11 mg/ml, or about 12 mg/ml, or about 13 mg/ml, or about 14 mg/ml, or about 15 mg/ml, or about 16 mg/ml, or about 17 mg/ml, or about 18 mg/ml, or about 19 mg/ml, or about 20, or about 21 mg/ml, or about 22 mg/ml, or about 23 mg/ml, or about 24 mg/ml, or about 25 mg/ml, or about 1 mg/ml to about 25 mg/ml, or 2.5 mg/ml to about 25 mg/ml, or about 4 mg/ml to about 19 mg/ml, or about 5 mg/ml to about 18 mg/ml, or about 6 mg/ml to about 17 mg/ml, or about 7 mg/ml to about 16 mg/ml, or about 8 mg/ml to about 15 mg/ml.

In some examples, the cell culture system may further comprise a solution suitable for supporting the growth of a cell of interest. For example, the solution may include, but is not limited to, buffered saline (such as Phosphate Buffered Saline (PBS)), cell culture media, alginate solution (such as 0.5% alginate solution), growth factors, nutrients, antibiotics, and the like. Thus, in such examples, the cell culture system comprises fungal derived protein and solution in a 1:1, or 1:1.5, or 1:2, or 1:2.5, or 1:3, or 1:3.5, or 1:4, or 1:4.5, or 1:5, or 1:5.5, or 1:6, or 1:6.5, or 1:7, or 1:7.5, or 1:8, or 1:8.5, or 1:9, or 1:9.5, or 1:10, or 1:10.5, or more (fungal derived protein: solution). In such examples, the cell culture system comprises fungal derived protein and solution in at least 1:1, or at least 1:1.5, or at least 1:2, or at least 1:2.5, or at least 1:3, or at least 1:3.5, or at least 1:4, or at least 1:4.5, or at least 1:5, or at least 1:5.5, or at least 1:6, or at least 1:6.5, or at least 1:7, or at least 1:7.5, or at least 1:8, or at least 1:8.5, or at least 1:9, or at least 1:9.5, or at least 1:10, or at least 1:10.5, or more (fungal derived protein:solution)

In yet another aspect, there is provided a method of forming a cell culture matrix from a fungus, comprising extracting fungal derived protein from the fungi. As described in the Experimental Section below, the fungal derived protein as described herein can be extracted from a fungus using a typical protein extraction procedure. In some examples, the fungal derived protein as described herein may be extracted under a protein extraction procedure known in the art. In some examples, the fungal derived protein as described herein may be extracted by processing the fungus such that it forms fine particles. For example, the fungus may be mechanically pulverized, grounded (such as manually or using a ball mill), or sheared.

In some examples, the fungal derived protein as described herein may be extracted under a protein extraction procedure under slightly basic conditions as known in the art. In some examples, the fungal derived protein as described herein may be extracted using any aqueous medium, such as, but is not limited to, deionized water, buffer (such as TRIS buffer) and the like. In some examples, the fungal derived protein as described herein may be extracted under a protein extraction procedure under slightly basic conditions (such as, but is not limited to, 0.01 M TRIS-HCl, 5 mM MgCl₂). In some examples, the fungal derived protein as described herein may be extracted and/or isolated using a pH 6, 2-morpholinoethanesulfonic acid (MES) buffer.

In some examples, the method as described herein may include extraction that comprises aggregation (precipitation) of the protein to form self-aggregating precipitate. For example, as shown in the Experimental Section, spontaneous precipitation of solid particulates from fungal extracts was observed when the extract solution was left standing at room temperature for a prescribed period. However, without wishing to be bound by theory, the inventors surprisingly found that boiling the extract for a prescribed period could also produce the desired precipitation of the fungal derived protein. In contrast to general knowledge, the inventors found that heating the protein fungal extract, which advantageously remove higher structures, still retains primary structures and self-adhesive sequence (such as RGD motif and/or YF motif) that are useful and needed in cell-adhesion. In some examples, the extraction step comprises a fungal derived protein denaturation step that causes aggregation to occur, optionally wherein the denaturation step retains the primary sequence structure of the fungal derived protein, optionally the denaturation step retains the cell-adhesive sequence (such as RGD motif and/or YF motif) of the fungal derived protein. Thus, in some examples, the aggregation (precipitation) step may comprise boiling the protein and/or incubating the protein in a condition that allows self-aggregating precipitate. In some example, the condition that allows self-aggregating precipitate to occur includes but is not limited to providing the protein in a concentration capable of inducing aggregation. In some examples, the precipitation (or aggregation) step may comprise providing the extracts for denaturation in a concentration of 0.4 mg/mL or more, or between 0.4 mg/mL to 10 g/mL, or about 0.4 mg/mL, or about 0.5 mg/mL, or about 0.6 mg/mL, or about 0.7 mg/mL, or about 0.8 mg/mL, or about 0.9 mg/mL, or about 1 mg/mL, or about 1.5 mg/mL, or about 2 mg/mL, or about 2.5 mg/mL, or about 3 mg/mL, or about 3.5 mg/mL, or about 4 mg/mL, or about 4.5 mg/mL, or about 5 mg/mL, or about 6 mg/mL, or about 7 mg/mL, or about 8 mg/mL, or about 9 mg/mL, or about 10 mg/mL.

In some examples, the precipitation (or aggregation) step may comprise heat treating the extracts for about 1 min to about 30 mins. In some examples, the precipitation step may comprise boiling the extracts for at least 1 min, at least 2 mins, at least 3 mins, at least 4 mins, at least 5 mins, at least 6 mins, at least 7 mins, at least 8 mins, at least 9 mins, at least 10 mins, at least 11 mins, at least 12 mins, at least 13 mins, at least 14 mins, at least 15 mins, at least 16 mins, at least 17 mins, at least 18 mins, at least 19 mins, at least 20 mins, at least 25 mins, or at least 30 mins. In some examples, the precipitation step may comprise boiling the extracts for about 2 mins to 30 mins, 3 mins to 27 mins, 4 mins to 25 mins, 5 mins to 20 mins.

In some examples, as would be appreciated by a person skilled in the art, the time required to heat the extracts may be dependent on the temperature of heat treatment of extracts. That is, in some examples, when the precipitation (or aggregation) step comprises heating the extracts at a high temperature, the time required to heat the extract may be shorter than when the precipitation (or aggregation) step comprise heating the extracts at a lower temperature.

In some examples, the precipitation (or aggregation) step may comprise heat treating the extracts from about 50° C. to about 350° C., or at least 50° C., or at least 60° C., or at least 70° C., or at least 80° C., or at least 90° C., or at least 100° C., or at least 110° C., or at least 120° C., or at least 130° C., or at least 140° C., or at least 150° C., or at least 160° C., or at least 170° C., or at least 180° C., or at least 190° C., or at least 200° C., or at least 210° C., or at least 220° C., or at least 230° C., or at least 240° C., or at least 250° C., or at least 260° C., or at least 270° C., or at least 280° C., or at least 290° C., or at least 300° C., or at least 310° C., or at least 320° C., or at least 330° C., or at least 340° C., or at least 350° C.

In some examples, on formation of self-aggregating precipitate, the method may further comprise the step of collecting the precipitate and washing of the precipitate.

In some examples, the method of forming the cell culture matrix as described herein may further comprise allowing and/or inducing and/or providing the correct condition for the fungal derived protein and/or cell culture matrix as described herein to form (or be used as) a gel (such as hydrogel and/or particulate gels) and/or coating. In some examples, the condition for the fungal derived protein and/or cell culture matrix to form gel may be as described herein above. In some examples, the condition may comprise providing the fungal derived protein and/or cell culture matrix in the right concentration to form a gel and/or coating. In some examples, the condition may further comprise increasing the ionic strength (such as providing buffered saline) of the solution and/or medium comprising the fungal derived protein and/or cell culture matrix. In some examples, the condition may further include adjusting the pH of the solution and/or medium and/or environment of the fungal derived protein and/or cell culture matrix. In some examples, the condition may further include the addition of cations (such as polycations). In some example, the condition may be the addition of a solution of chitosan in acetic acid.

Also disclosed is a method of forming a cell culture matrix comprising providing the fungal derived protein and/or cell culture matrix as described herein to form a gel (such as hydrogel) and/or a coating.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications related to structural and/or functional changes may be made without deviating from the scope of the invention. Example embodiments are not necessarily mutually exclusive as some may be combined with one or more embodiments to form new exemplary embodiments. The example embodiments should not be construed as limiting the scope of the disclosure.

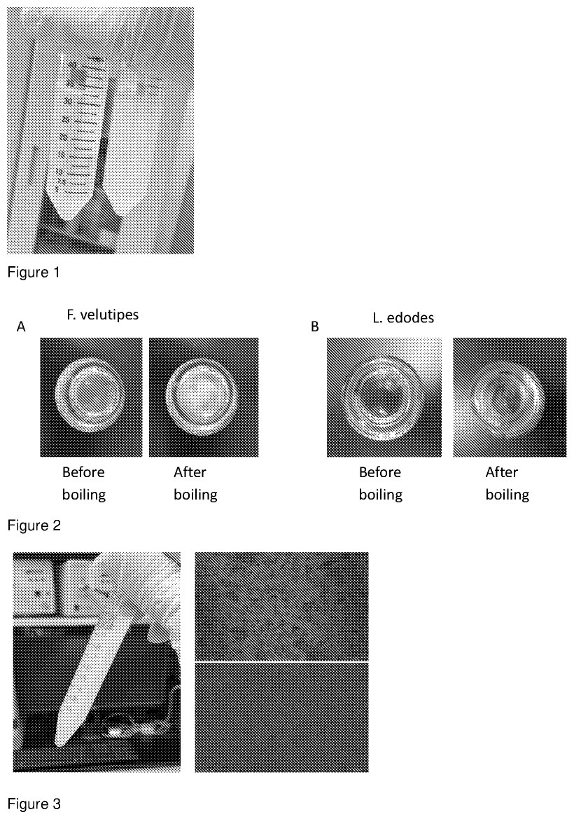

FIG. 1 shows the comparison of fresh extract from F. velutipes and extract after standing at room temperature (25° C.) for several days. Spontaneous precipitation had occurred to give a suspension of white particulates.

FIG. 2 shows photos of boiling of extracts from mushroom extracts yield particulate suspensions.

FIG. 3 shows photos of extracts from mushroom extracts. (A) Particles can be dispersed easily in water to give a particulate suspension. Light microscope images of (B) clusters of particulates in boiled solution; (C) uniformly dispersed particles after isolation by centrifugation and brief vortexing.

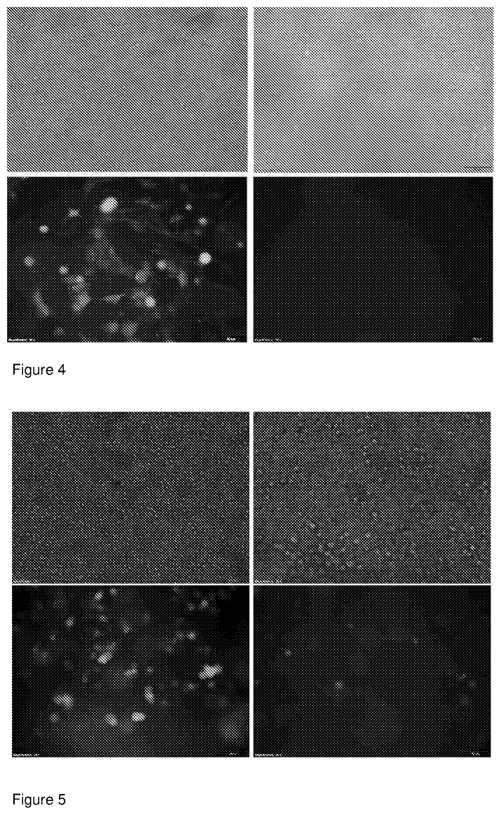

FIG. 4 shows microscopic images of cells in F. velutipes particles. (A) C2C12 cells entrapped in a matrix of F. velutipes particles upon mixing 25 μL of a cell suspension in DMEM media (with serum) and 10 μL of particle suspension. Cells appear in different focal planes within the gel; Live-dead assay after 4 hours showing good viability of the entrapped cells: (B) bright field image; (C) Calcein staining for live cells, shows the spreading of cells within the gel matrix; (D) Ethidium homodimer staining for dead cell nuclei.

FIG. 5 shows microscopic images of cells in L. edodes particles. (A) C2C12 cells entrapped in a matrix of L. edodes particles upon mixing 10 μL of particle suspension and 50 μL of a cell suspension in DMEM media (with serum). Live-dead assay after 7 hours showing good viability of the entrapped cells: (B) bright field image; (C) Calcein staining for live cells; (D) Ethidium homodimer staining for dead cell nuclei. The gel matrix picks up some of the red stain.

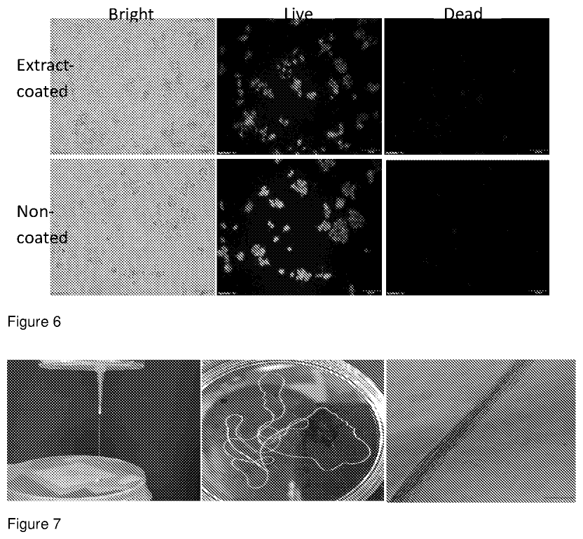

FIG. 6 shows light microscope images of MCF7 cells seeded on F. velutipes particle-coated and non-coated plates after 20 h of culture.

FIG. 7 shows photos of various fibres drawn from F. velutipes particle. (A) Fibre drawn from the interface between chitosan and alginate-F. velutipes particle solutions. Note the distinct interface boundary between the two solutions; (B) Fibre being washed in deionized water; (C) Fibre exhibits a uniform appearance and dispersion of particles.

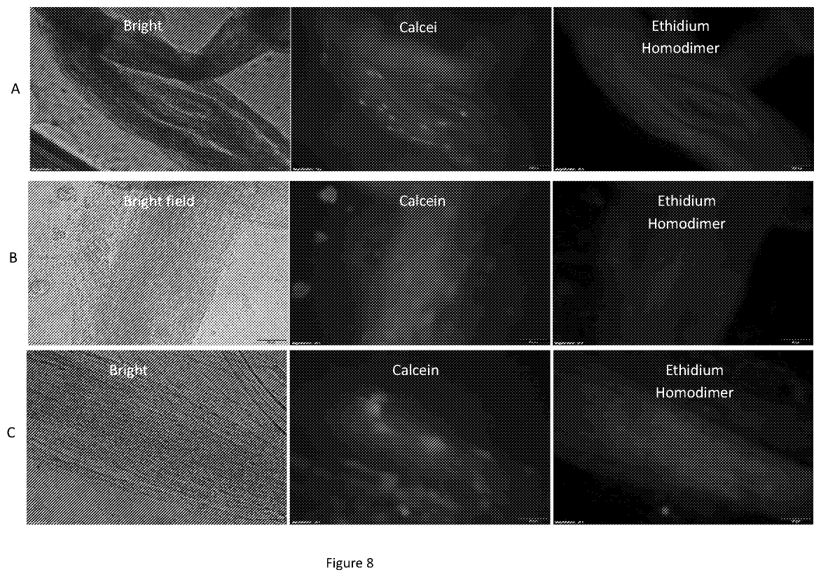

FIG. 8 shows microscopic behaviours of cell lines on F. velutipes particles. Good attachment of C2C12 mouse myoblast cell line on chitosan-alginate fibre incorporated with F. velutipes protein particles. Panel A: At lower magnification; Panel B: At higher magnification, showing cells packed closely on fibre surface; Panel C: At higher magnification, showing good cell spreading with formation of cellular processes.

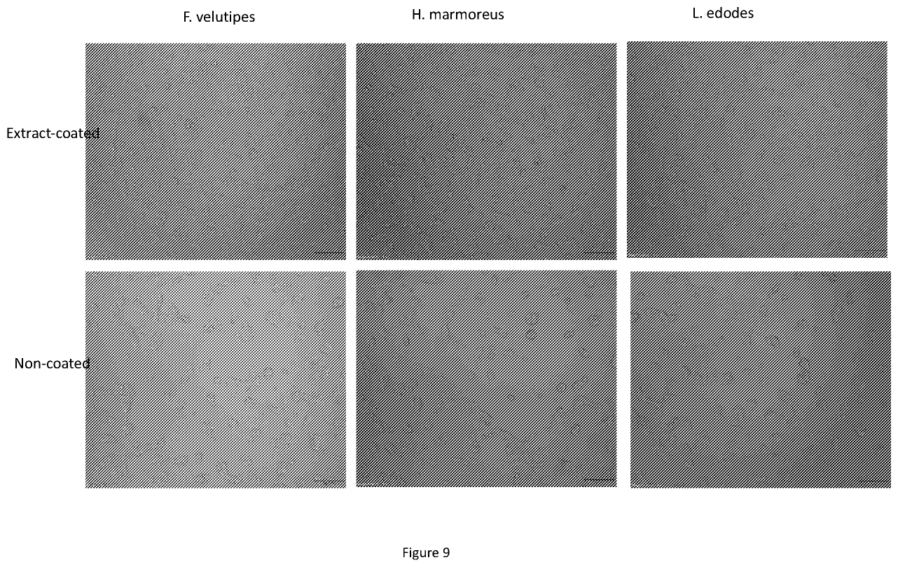

FIG. 9 shows light microscope images of MCF7 cells seeded on mushroom extract-coated and non-coated plates at 18 h.

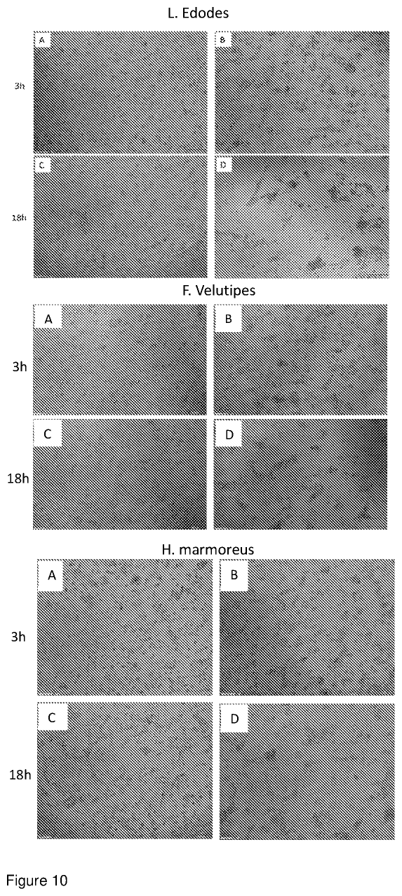

FIG. 10 shows light microscope images of primary human skeletal muscle cells seeded on mushroom extract-coated (A, C) and non-coated (B, D) plates after 3 h and 18 h of culture.

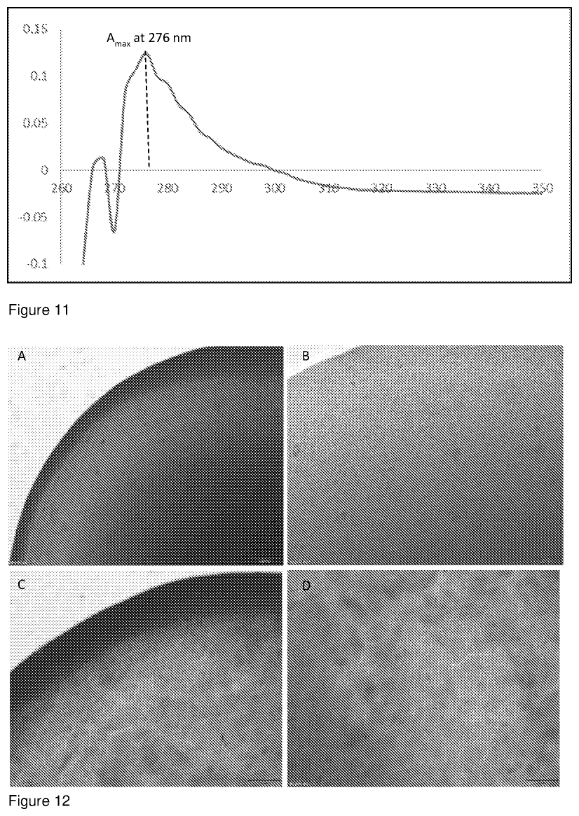

FIG. 11 shows ultraviolet subtraction spectrum of extract from F. velutipes before and after spontaneous precipitation. Aₘₐₓ at 276 nM indicates protein being precipitated out of the solution.

FIG. 12 shows the addition of phosphate buffered saline (PBS) to L. edodes particulate suspension leads to particle aggregation and formation of a particulate gel. (A,B) Prior to adding PBS; (C,D) Immediately after addition of PBS.

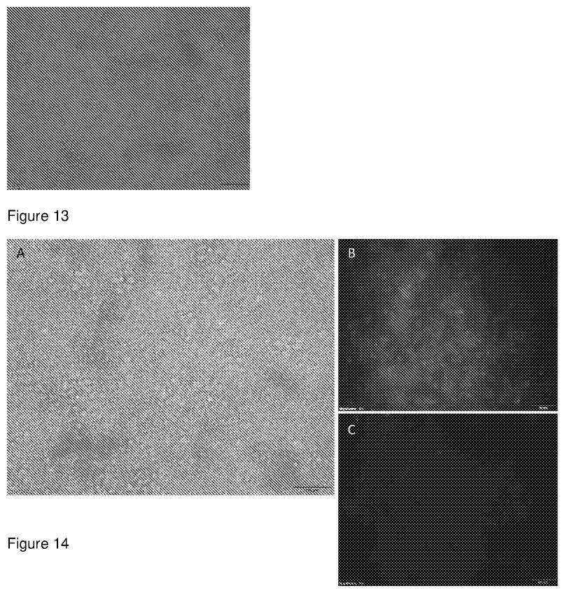

FIG. 13 shows the formation of a gel upon addition of 11 mg/mL F. velutipes particles to C2C12 cell suspension in DMEM media, after 10 mins.

FIG. 14 shows C2C12 cells cultured in F. velutipes particulate gel over 2 days. (a) bright field image; (b) Calcein staining for live cells; (c) Ethidium homodimer staining for dead cell nuclei.

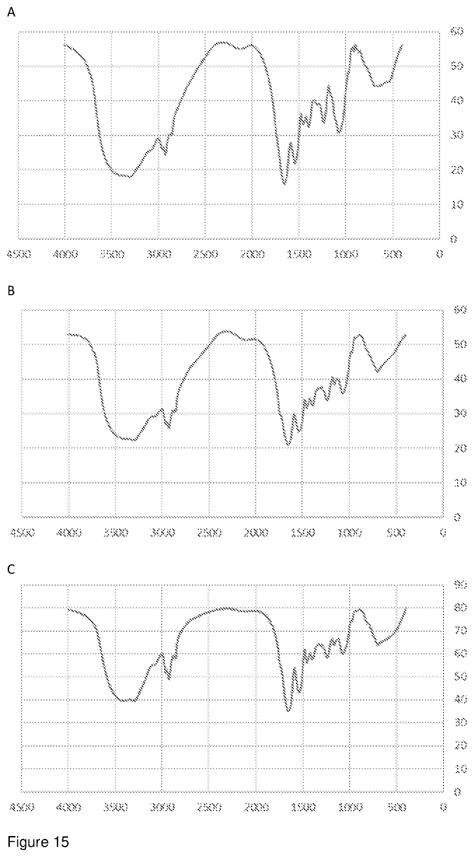

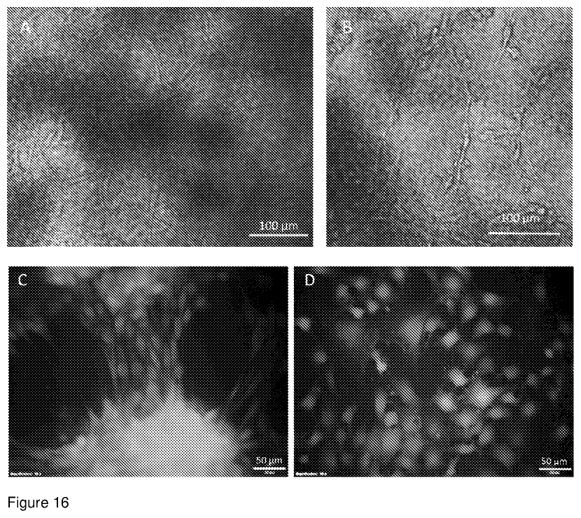

FIG. 15 shows the FT-IR spectra of cell-adhesive particles precipitated from F. velutipes (A, B) and L. Edodes (C). The y-axis and x-axis are % absorbance and wavelength (cm-1) respectively.

FIG. 16 shows particulate gel from F. velutipes promotes muscle cell alignment.

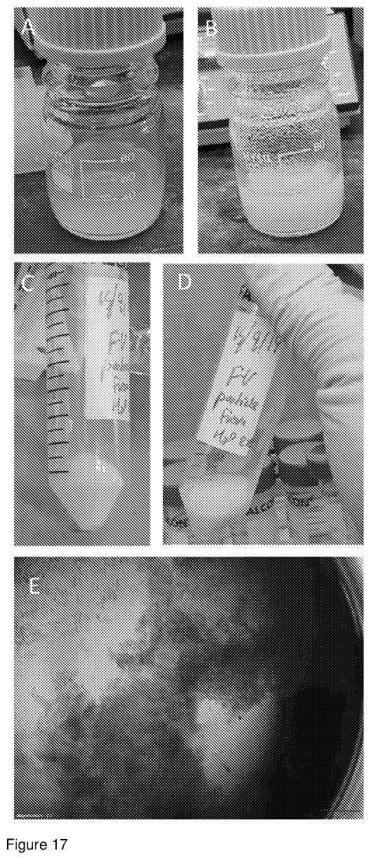

FIG. 17 shows water extract of F. velutipes (A) before, and (B) after boiling; (C) Isolated precipitate; (D) Precipitate dispersed uniformly in water forming a white, milky suspension; (E) Addition of media to particle suspension results in a particulate gel.

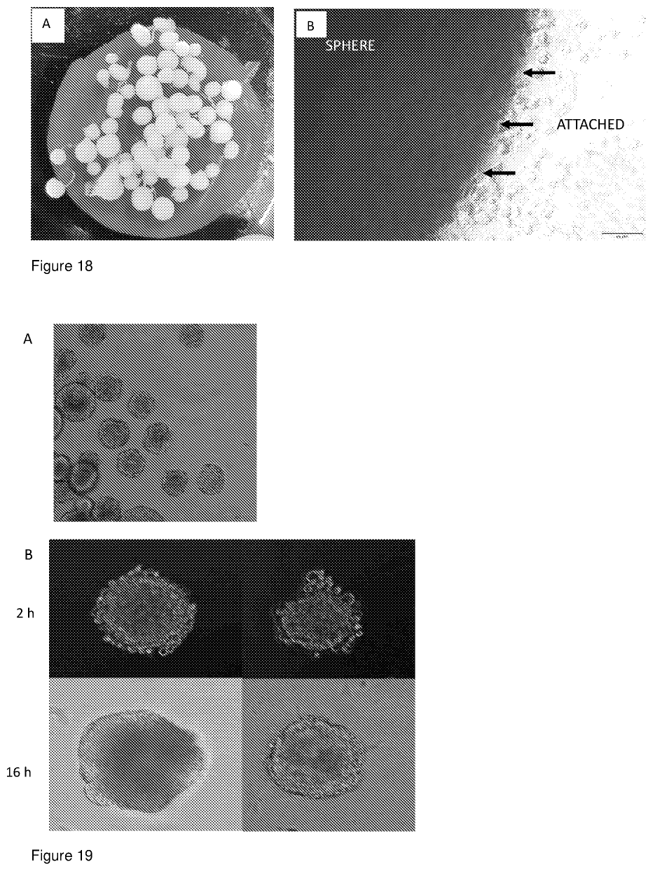

FIG. 18 shows photographs of (A) alginate spheres incorporating FV particles; and (B) attachment of C2C12 mouse myoblasts to FV-alginate incorporated spheres.

FIG. 19 shows photographs of (A) FV-alginate-coated chitosan microspheres; and (B) attachment of C2C12 cells to the microspheres.

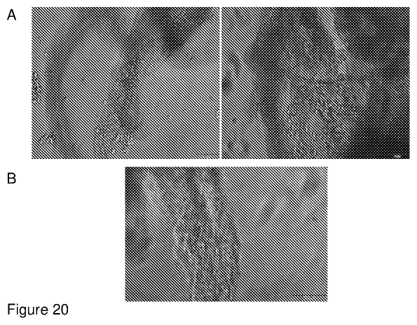

FIG. 20 shows photographs of (A) C2C12 cells attached to FV particle incorporated water-soluble chitin (WSC)-alginate fibers after 1 h; and (B) cells observed to have spread on the fiber surface at 24 h.

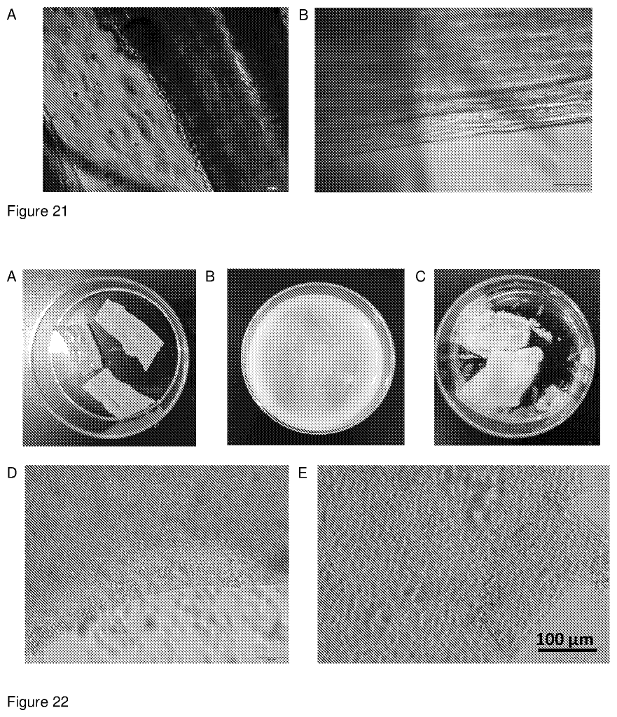

FIG. 21 shows photographs of (A) attachment of C2C12 cells on FV-alginate coated cellulose fibers; and (B) cells had spread and elongated along the axis of the fibers after 2 days.

FIG. 22 shows photographs of (A) planar constructs from chitosan and sodium alginate; (B) immersion of the constructs in FV-alginate suspension; (C) coagulation of FV-alginate suspension to form films; and C2C12 cells seeded on the films that showed (D) good attachment after 1 h, and (E) spreading after 1 day.

### EXPERIMENTAL SECTION

Materials and Methods

Materials

The three fungal species, Flammulina velutipes, Lentinus edodes and Hypsizigus marmoreus are edible mushrooms which were procured from local retailers. 2-amino-2(hydroxymethyl)-1,3-propanediol hydrochloride (TRIS-HCl), magnesium chloride hexahydrate (MgCl₂.6H₂O) and 2-morpholinoethanesulfonic acid (MES) were obtained from Merck, Darmstadt, Germany. Breast cancer cell line, MCF7 and mouse myoblast cell line, C2C12 were obtained from American Type Culture Collection (ATCC), VA, USA. Primary human skeletal muscle cells were obtained from Promocell GmbH, Heidelberg, Germany.

Preparation of Fungal Extracts

The mushroom pileus and stipes were frozen at −80° C. for at least an hour, followed by grounding with mortar and pestle to achieve a slurry. 25 mL of buffer (0.01 M TRIS-HCl, 5 mM MgCl₂) was added to 14 g of mushroom wet mass. The suspension was sonicated (Vibracell VCX130PB Ultrasonic Processor) for 30 min on ice, followed by centrifugation at 10,000 rpm for 15 min using an Eppendorf refrigerated centrifuge at 4° C. The supernatant was decanted into a fresh tube or blue cap bottle and designated as the mushroom extract.

Protein Particle Precipitation

Example 1: 28 g of ground F. velutipes was extracted using 25 mL of TRIS buffer. From the 35 mL of resulting extract, 15 mL was decanted into a 50 mL Blue Cap bottle and heated on a Heidolph hot-plate set at 150° C. Within 10 min, boiling of the solution had occurred, and a white precipitate had appeared in the solution, which was isolated by centrifugation at 7000 rpm for 5 min. The remaining 20 mL of extract was allowed to stand at room temperature (25° C.). Within 48 hours, a white turbidity had appeared in the solution, which could be isolated as the same precipitate.

Example 2: 14 g of ground L. edodes was extracted using 25 mL of TRIS buffer. The resulting extract was decanted into a 50 mL Blue Cap bottle and heated on a Heidolph hot-plate set at 200° C. Within 20 min, boiling of the solution had occurred, and a white precipitate had appeared in the solution.

Example 3: 20 g of ground F. velutipes or L. edodes was extracted using 25 mL of TRIS buffer. The resulting extract was decanted into a 50 mL Blue Cap bottle and heated on a Heidolph hot-plate set at 200° C. Within 10 min, boiling of the solution had occurred, and a white precipitate had appeared in the solution, which was centrifuged at 7000 rpm for 5 min. The yield of precipitate (particles) was measured to be 60-70 mg per 20 g of mushroom wet mass.

Characterization of Protein Particles

UV spectrophotometry. The uv-spectrum for a solution of F. velutipes extract and the supernatant of the same solution after spontaneous precipitation was measured in the uv wavelength range. The subtraction spectrum indicated that a protein compound had been precipitated from the solution, with maximum absorbance at 276 nm (FIG. 11).

Formation of Hydrogels by Particle Aggregation and 3D Mammalian Cell Culture

Phosphate buffered saline (PBS) was added to L. edodes or F. velutipes particle suspension on a coverslip. Aggregation of the particles was observed under the light microscope, leading to the formation of a particulate gel (FIG. 12).

10 μL of F. velutipes or L. edodes particle suspension (8-15 mg/mL) was added to 25 μL or 50 μL of DMEM serum-containing media. A particulate gel was formed (FIG. 13).

For 3D cell culture, 25 μL or 50 μL of C2C12 cell suspension in serum-containing DMEM media was added to a 96-well plate. 10 μL F. velutipes or L. edodes particle suspension (8-15 mg/mL) was then added to the cell suspension and immediately mixed by tituration. Formation of a slightly turbid particulate gel was observed, entrapping the cells (FIG. 14). The C2C12 cell suspension could also be added to the particle suspension in the well, followed by mixing.

Results

Isolation and Characterization of Particles

A typical protein extraction procedure under slightly basic conditions (0.01 M TRIS-HCl, 5 mM MgCl₂) was employed. However, it was found that the particles could also be isolated using a pH 6 2-morpholinoethanesulfonic acid (MES) buffer.

Upon standing for two days at room temperature, spontaneous precipitation of solid particulates from the extract from F. velutipes occurred (FIG. 1). Precipitation took a substantially longer time (in the order of weeks) for the extracts from the other two mushroom species, and to lesser degree. After the F. velutipes suspension was centrifuged to isolate the precipitate, and the supernatant allowed to stand further, more precipitate formed in the supernatant with time.

It was found that precipitation could be induced to obtain the same particulate suspension by boiling the F. velutipes and L. edodes extracts for 5-20 min (FIG. 2a). However, the H. Marmoreus extract could not be induced to precipitate by boiling alone (evaporation to concentrate the solution was required). For the extracts from F. velutipes and L. edodes, boiling is postulated to result in denaturation and unfolding of the RGD-containing protein, leading to its self-aggregation to form particles. In this case, protein denaturation is desirable as the cell-adhesive function of the RGD motif, being in the primary sequence, would not be lost, while the protein would lose other native functions that depend on its higher level structures (The latter functions may not be desirable for a general matrix for 3D cell culture). After being centrifuged to obtain a pellet, the particles could be dispersed easily by brief vortexing (˜1 min) in deionized water (FIG. 3). To further purify the particles for characterization, they were dialyzed against several changes of deionized water overnight.

By zetasizer analysis, the particles from F. velutipes were found to possess an average diameter of 1050 nm and zeta potential of −28 mV, while the particles from L. edodes measured 960 nm with a zeta potential of −20 mV. The relatively high negative surface charge of the particles leads them to repel each other and prevents aggregation, explaining the easy dispersibility and stability of the particles in aqueous solution.

Particulate Gels and Coatings

When the suspension of F. velutipes particles (8-15 mg/mL) was mixed with PBS or media (typically at a 1:2.5 or 1:5 ratio), aggregation of the particles occurred, resulting in a particulate gel. When a cell suspension was used in place of media, the cells were entrapped in the 3D particulate matrix (FIG. 4). Additional medium could be dispensed on top of the hydrogel, with regular changes as necessary for continuous cell culture. It was observed that cultured cells could spread out and extend processes in the hydrogel matrix, attributed to the presence of the RGD motif in the protein of the particles (FIG. 4c). In the same way, cells could be cultured in gels formed from L. edodes particle suspensions (8-15 mg/mL) (FIG. 5). For both gel types, cells remained viable when cultured continuously over the longer term.

The particulate gels could be redispersed in water, an advantage for the passaging of cells and their retrieval for analysis, e.g. for RT-PCR.

The particles from the mushroom extracts could also be used for coating of cell culture surfaces. MCF7 cells adhered on F. velutipes particle-coated plates and demonstrated good viability (FIG. 6).

Incorporation of Particles into Chitosan-Alginate Fibres and Cell Adhesion on the Fibers

1 mL of a 12 mg/mL suspension of F. velutipes particles in deionized water was centrifuged at 7000 rpm for 5 min in an Eppendorf vial. 700 μL of supernatant above the pellet was withdrawn using a pipette. The pellet was redispersed in the remaining 300 μL of water by vortexing for several minutes. 3 mg of sodium alginate (medium molecular weight, Sigma-Aldrich) was then added to this particulate suspension and vortexed further for at least 10 min to completely dissolve the alginate.

By the process of interfacial polyelectrolyte complexation, the resulting mixture (40 mg/mL particles and 1% alginate) was then used to draw fibre against 1% chitosan (high molecular weight, Sigma-Aldrich) in 0.15M HOAc. A linear motor with an attached pipette tip at a vertical upward speed of 40 mm/min was employed to draw fibre at the interface between the two solutions (FIG. 7A).

The fibres were washed with water in a petri dish (FIG. 7B), after which the samples were transferred to wells of a 96-well tissue culture plate. Light microscope observation indicated that the particles were well-distributed in the fibres (FIG. 7C). A C2C12 mouse myoblast cell line was then seeded on these fibres. A LIVE/DEAD Viability/Cytotoxicity Kit (Thermo Fisher Scientific) was used to visualize cell attachment to the fibres after 72 h of culture. Excellent adhesion of cells onto the fibres was observed (FIG. 8).

Cell Adhesive Properties of the Fungal Extracts

It was found that all three fungal extracts (from L. edodes, F. velutipes and H. marmoreus), prior to precipitation, could mediate cell attachment. Cells attached and spread out to a higher degree on extract-coated PS microtiter wells, compared to the non-coated control wells, for both MCF7 cells (FIG. 9), and primary human skeletal muscle cells (FIG. 10).

In view of the above, the ability to formulate hydrogels from protein particles induced to precipitate from fungal extracts introduces a spectrum of new materials for 3D cell culture. Furthermore, the use of fungal, as opposed to animal-derived materials as cell-adhesive extracellular matrices for mammalian cells, may be environmentally more sustainable in the long term, and holds particular promise for Clean Meat applications. The three species of mushroom presented as examples in this work are all edible mushroom species from the order Agaricales (gilled mushrooms). Given that there are an estimated 13,000 species of fungi in this order alone, there is a high possibility that other candidates exist which may yield cell-adhesive mushroom extracts for biomaterials and cell culture applications.

### Example A1

To further confirm the proteinaceous nature of the cell-adhesive particles extracted from the fungi, Fourier Transform-Infra-Red (FT-IR) spectra of F. Velutipes particles obtained by TRIS buffer extraction (A) and water extraction (B) were obtained, in addition to that of L. Edodes particles obtained by TRIS buffer extraction (C).

All three FT-IR spectra were similar (see FIG. 15), and showed characteristics of protein samples, with NH stretching vibrations at 3300 cm⁻¹ and 3070 cm⁻¹, amide I vibration at 1650 cm⁻¹, amide II vibration at 1550 cm⁻¹, and amide III vibrations between 1200 and 1400 cm⁻¹. A relative strong absorption band was observed at 1100 cm⁻¹, especially for the TRIS buffer-extracted F. Velutipes particles, which is believed to be due to a relatively high content of the amino acid, tryptophan in the proteins.

### Example A2

It was demonstrated that the fungal-derived cell adhesive matrices were able to promote alignment of C2C12 mouse myoblast cells.

15 μL of F. Velutipes particle suspension in water (concentration of approx. 10-15 mg/mL) was mixed with 25 μL C2C12 suspension (˜1.8×10⁵ cells/mL) in a 96 well-plate, followed by an additional 25 μL of DMEM media (10% FBS) after 50 min. Control wells where 15 μL of sterile water was used in place of the particles were also prepared (n=4). At the end of 1 day, wells were topped up with 25 μL DMEM (without FBS).

After 1 day in culture, cells in the wells containing particles had begun to align themselves (FIG. 16A). These aligned cells that appeared to connect clusters of myoblasts persisted after 3 days of culture, as demonstrated by a Live Dead assay, which also indicated excellent cell viability (FIGS. 16 B, and C). In contrast, cells in the control wells were viable but not aligned (FIG. 16 D).

Alignment of muscle cells is an important step that precedes myotube formation (Zhao Y et al. Biotechnol Bioeng. 102(2) (2009) 10.1002/bit.22080). Thus, promotion of muscle cell alignment by the fungal-derived protein particles may be advantageous for muscle cell differentiation and subsequent maturation.

### Example A3

Extraction of F. velutipes particles could be carried out using deionized water, i.e. TRIS or other buffer is not required.

20 g of ground F. velutipes was extracted using 25 mL of deionized water. The resulting extract was decanted into a 100 mL media storage bottle and heated on a Heidolph hot-plate set at 200° C. Within 10 min, boiling of the solution had occurred, and a white precipitate had appeared in the solution, which was isolated by centrifugation at 7000 rpm for 5 min (see FIG. 17).

As for the TRIS buffer-extracted protein particles, particulate gels could be prepared in the same way using the water-extracted protein particles.

### Example A4

The same protein extraction and precipitation procedure, as presented in this TD, was applied on another edible fungal species, Grifola Frondosa. As for the case of Hypsizygus Marmoreus, boiling of the extract alone did not yield any protein particles. It is believed that this is an argument in favour of an inventive step, as the boiling procedure is not expected to yield precipitate (protein particles) for any fungal extract in general.

The inventors also found that protein extraction and precipitation used heat that is much higher than what is typically used. In particular, general knowledge in the art holds that low temperatures (such as 30° C.) should be used to obtain protein from fungal species, and the fungus should not be heated to temperatures of 100° C. or higher, as the latter would result in protein denaturation. However, in contrast to general knowledge, the inventors found that heating the protein fungal extract results in particles that retain cell-adhesive function, which suggests that heating advantageously removes higher structures but still retains primary structures and cell-adhesive sequences such as the RGD motif.

### Additional Examples

The inventors of the present disclosure further found that a suitable and effective approach to imbue cell-adhesive factors into the biomaterial forms was by bulk-incorporation or coating of a suspension of particles from F. Velutipes in alginate solution (FV-alginate), followed by crosslinking with calcium.

In the following, examples are presented of bulk-incorporation and coating of FV-alginate fibers in/on fiber, sphere and film forms of various biomaterials. In each case, the precipitate that had been obtained by boiling the F. Velutipes extract and centrifuging at 7000 rpm for 5 min (see Examples 1-3 above) was used as a starting material. The precipitate could be further washed with water by centrifugation and stored either as the wet precipitate or a suspension in water. These are referred to as ‘FV precipitate’ or ‘ FV particle suspension’ respectively.

### Example A5

FV-Alginate Incorporated Spheres

The FV particle suspension was centrifuged down at 7000 rpm for 5 min. The supernatant was decanted and 500 μL of deionized water was used to disperse the resulting pellet. 2 mL of 3% alginate (low molecular weight, Sigma-Aldrich) was added to this suspension and titurated/vortexed till the particles were uniformly dispersed (FV-alginate suspension). The suspension was drawn into a 3 mL syringe using a 21½ G needle. By gently depressing the plunger to produce tiny droplets while immersing and rapidly withdrawing the syringe needle in and out of 10 mL 5% (w/v) CaCl₂) solution contained in a 50 mL beaker, alginate spheres incorporating FV particles were obtained. The particles were washed and sterilized in 70% ethanol in a 24-well plate for 3 h, washed with phosphate buffered saline (PBS) and transferred to fresh wells. A suspension of C2C12 mouse myoblasts in DMEM/10% FBS (>10⁶ cells/mL) were seeded into these wells.

Results

Spheres were formed with diameters ranging from ˜1-2 mm in diameter (FIG. 18A). No observable leaching of FV particles occurred from the spheres. 2.5 h after cell seeding, C2C12 mouse myoblast attachment was observed on the spheres (FIG. 18B).

### Example A6

FV-Alginate Coated Chitosan Microspheres

Chitosan microspheres (˜100 μm in diameter) were prepared according to a water-in-oil microemulsion method. 500 μL of 3% FV-alginate suspension was added to 500 μL of a microsphere suspension in a 1.5 mL centrifuge tube. After 10 min of standing, the microspheres had sedimented to the bottom of the tube. 500 μL of the supernatant was removed and replaced with deionised water. The above step was repeated 4 times, allowing at least 2 min for microsphere deposition between each repeat. At the end of the last repeat, the supernatant was clear, indicating complete removal of the FV-alginate particles. The supernatant was removed until approx. 50 μL remained over the deposited microspheres, 500 μL 5% (w/v) CaCl₂) solution was added and the suspension was titurated several times. The coated microspheres were allowed to deposit, then the CaCl₂) solution was removed and replaced with 75% ethanol. The microspheres were transferred to a 24-well plate and allowed to stand for 1 h, after which they were washed with phosphate-buffered saline. The appearance of the FV-alginate-coated chitosan microspheres is shown in FIG. 19A. A suspension of C2C12 mouse myoblasts in DMEM/10% FBS (>10⁶ cells/mL) were then seeded onto the microspheres. After 2 h, the microspheres were transferred into fresh wells for observation.

Results

Within 2 h of cell seeding, attachment of C2C12 cells to the microspheres had occurred. Large numbers of attached cells could be observed on the periphery of the microspheres. After overnight (16 h) culture, the cells had spread out onto the microspheres, whose surfaces had transited from a more granular appearance where individual cells could be discerned, to a smoother, homogenous surface with bumps. (FIG. 19B).

### Example A7

FV Particle Incorporated Water-Soluble Chitin (WSC)-Alginate Fibers

FV precipitate was resuspended in 5 mL deionised water by vortexing for at least 1 min. 1 mL of the particle suspension was transferred to a 1.5 mL centrifuge tube. The suspension was centrifuged at 7000 rpm for 5 min and the resulting pellet was dispersed by vortexing in 200 μL 3% alginate (low MW, Sigma-Aldrich). Fiber was drawn by a process of interfacial polyelectrolyte complexation using 10 μL of the FV-alginate particle suspension and 0.5% WSC. The drawn fiber was immediately immersed into 5% (w/v) CaCl₂) solution contained in a petri dish. The fiber was rinsed with deionised water and sterilized with 70% ethanol for at least 1 h. The ethanol solution was replaced with PBS. The PBS solution was removed and a suspension of C2C12 mouse myoblasts in DMEM/10% FBS (>10⁶ cells/mL) was transferred to the well containing the fiber. After 1 h, the fiber (with attached cells) was transferred to a fresh well.

Results

No leaching of FV particles from the fiber occurred during the washing process. At 1 h, a substantial number of cells was observed attached to the fiber (FIG. 20A). After 24 h, these cells appeared to have spread out on the fiber surface (FIG. 20B). A Live Dead assay showed that most of the attached cells retained good viability.

### Example A8

FV-Alginate Coated Cellulose Fibers

Preparation of FV-alginate suspension: FV precipitate was resuspended in 5 mL deionised water by vortexing for at least 1 min. 1 mL of the particle suspension was transferred to a 1.5 mL centrifuge tube. The suspension was centrifuged at 7000 rpm for 5 min and the resulting pellet was dispersed by vortexing in 200 μL 3% alginate (low MW, Sigma-Aldrich).

Cellulose fibers were immersed in 25% NaOH solution in a petri dish overnight. The fibers were washed thrice with deionised water, soaked in water for 30 min, then immersed in 5% w/v CaCl₂ solution for at least 10 min. By centrifugation at 3500 rpm for 5 min, the fibers were separated from the supernatant and washed with deionised water. Subsequently, they were immersed in the FV-alginate suspension, as prepared above. After at least 30 min of standing, the suspension was removed and deionised water added to wash the fibers. This process was repeated until the supernatant was clear. The fibers were transferred to a 24-well plate and immersed in 70% ethanol for at least half hour. The ethanol solution was then replaced with PBS, followed by a C2C12 mouse myoblast suspension in DMEM/10% FBS (>10⁶ cells/mL). After 2 h, the fibers (with attached cells) were transferred to fresh wells for observation.

Results

Good attachment of cells on the fibers was observed after 2 h (FIG. 21A). After 2 days of culture, the cells had spread and elongated along the axis of the fibers (indicated by arrows in FIG. 21B). At this time-point, a Live Dead assay indicated good viability of the cells.

### Example A9

FV-Alginate Films

FV alginate films can be obtained in the following way. Planar constructs were made by an interfacial polyelectrolyte complexation process using chitosan and sodium alginate (FIG. 22A). As these constructs had been calcium crosslinked using 25 mM calcium chloride during the process, they contained calcium solution. FV-alginate suspension (as prepared in Example A8) was added to a petri dish containing the constructs to completely immerse them (FIG. 22B). After several minutes, the FV-alginate suspension directly on top of the constructs had coagulated to form films due to crosslinking by calcium leaching out from the constructs, and these could be easily be peeled off (FIG. 22C). C2C12 cells that were seeded onto these films showed good attachment after 1 h (FIG. 22D) and spreading after 1 d (FIG. 22E).

### REFERENCES

1. Yasuda T, Ishihara H, Amano H, Shishido K. Generation of basidiomycetous hyphal cell-aggregates by addition of the Arg-Gly-Asp motif-containing fragment of high-molecular-weight cell-adhesion protein MFBA derived from the basidiomycete Lentinus edodes, Biosci Biotechnol Biochem. 61(9) (1997) 1587-9
2. Kondoh O, Muto A, Kajiwara S, Takagi J, Saito Y, Shishido K. A fruiting body-specific cDNA, mfbAc, from the mushroom Lentinus edodes encodes a high-molecular-weight cell-adhesion protein containing an Arg-Gly-Asp motif, Gene 154(1) (1995) 31-7
3. Sakamoto Y, Azuma T, Ando A, Tamai Y and Miura K. Characterization of proteins expressed abundantly in the fruit-body of Flammulina velutipes, Mycoscience 41 (2000) 279-282

1. A cell culture matrix comprising a fungal derived protein.

2. The cell culture matrix of claim 1 wherein the fungal derived protein comprises a cell-adhesive sequence, optionally, the fungal derived protein retains its primary sequence.

3. The cell culture matrix of claim 1, wherein the fungal derived protein is free of native functions.

4. The cell culture matrix of claim 1, wherein the fungal derived protein forms particles having an average diameter of about 500 nm to about 1600 nm.

5. The cell culture matrix claim 1, wherein the fungal derived protein forms particles having a zeta potential of about −10 mV to about −50 mV.

6. The cell culture matrix of claim 1, wherein the fungal derived protein extract forms a gel and/or a coating, optionally the fungal derived protein extract forms a hydrogel.

7. The cell culture matrix of claim 1, wherein the fungi are non-pathogenic fungi.

8. The cell culture matrix of claim 1, wherein the fungi are from the order of Agaricales, optionally from the division Basidiomycota.

9. The cell culture matrix of claim 1, wherein the fungi are species selected from the group consisting of Flammulina velutipes, Lentinus edodes, and Hypsizigus mannoreus.

10. The cell culture matrix of claim 1, wherein the cell culture matrix further comprises a biomaterial component, optionally the cell culture matrix further comprises one or more selected from the group consisting of alginate, chitosan, chitin, and cellulose fibres.

11. The cell culture matrix of claim 1, where in the cell culture matrix further comprises crosslinkers, optionally the crosslinker is calcium.

12. The cell culture matrix of claim 1, wherein the cell culture matrix forms a coating comprising alginate that is calcium crosslinked.

13. A composition comprising the cell culture matrix of claim 1.

14. A cell culture system comprising a cell culture matrix comprising a fungal derived protein.

15. (canceled)

16. A method of forming a cell culture matrix from a fungus, comprising extracting a fungal derived protein from the fungi.

17. The method of claim 16, wherein the extracting comprises aggregating the fungal derived protein to forma self-aggregating precipitate.

18. The method of claim 16, wherein the extracting comprises aggregating the fungal derived protein by boiling the fungal derived protein and/or incubating the fungal derived protein in a condition that allows self-aggregating precipitate.

19. The method of claim 16, wherein the extracting comprises a fungal derived protein denaturation step that causes aggregation to occur, optionally wherein the denaturation step retains the primary sequence structure of the fungal derived protein, optionally the denaturation step retains the cell-adhesive sequence of the fungal derived protein.

20. The method of claim 16, wherein the extracting comprises an aggregation step comprising heat treating the fungal derived protein for about 1 min to about 30 mins, optionally the aggregation step comprises heating the fungal derived protein from about 50° C. to about 350° C.
